(19) United States
(12) Patent Application Publication
(10) Pub. No.: US 2022/0081291 A1
(43) Pub. Date: Mar. 17, 2022

(54) PARALLEL REFORMING IN CHEMICAL PLANT

(71) Applicant: HALDOR TOPSØE A/S, Kgs. Lyngby (DK)

(72) Inventor: Peter Mølgaard MORTENSEN, Roskilde (DK)

(73) Assignee: HALDOR TOPSØE A/S, Kgs. Lyngby (DK)

(21) Appl. No.: 17/421,449

(22) PCT Filed: Feb. 27, 2020
(86) PCT No.: PCT/EP2020/055178

§ 371 Date: Jul. 8, 2021

(30) Foreign Application Priority Data
Feb. 28, 2019 (DK) PA 2019 00256

(51) Int. Cl. C01B 3/38; C01B 3/48; C01B 3/50; B01J 8/06; C01C 1/04; C07C 1/04; C07C 29/151

(52) U.S. Cl. CPC C01B 3/382; B01J 2208/00539; C01B 3/50; B01J 8/067; B01J 8/065; C01C 1/0417; C07C 1/0485; C07C 29/1518; C01B 2203/0233; C01B 2203/0244; C01B 2203/0283; C01B 2203/0495; C01B 2203/1241; C01B 2203/061; C01B 2203/068; C01B 2203/062; C01B 2203/085; C01B 2203/0811; C01B 2203/0866; C01B 2203/141; B01J 2208/00504; B01J 2208/00477; B01J 2208/00389; C01B 3/48

(57) ABSTRACT

A chemical plant including: a reforming section arranged to receive a feed gas comprising hydrocarbons and provide a combined synthesis gas stream, wherein the reforming section includes: an electrically heated reforming reactor housing a first catalyst, an autothermal reforming reactor in parallel with the electrically heated reforming reactor, wherein the reforming section is arranged to output a combined synthesis gas stream including at least part of the first and/or second synthesis gas streams, an optional post processing unit downstream the reforming section, a gas separation unit arranged to separate a synthesis gas stream into a water condensate and an intermediate synthesis gas, and a downstream section arranged to receive the intermediate synthesis gas and to process the intermediate synthesis gas to a chemical product and an off-gas. Also, a process for producing a chemical product from a feed gas comprising hydrocarbons.

### FIELD OF THE INVENTION

The present invention relates to a chemical plant and a process for producing a chemical product by heterogeneous catalysis of a feed gas comprising hydrocarbons. The invention relates particularly to a plant and a process for producing a synthesis gas, a plant and process for producing methanol, a plant and process for producing ammonia and a plant and process for producing a mixture of higher hydrocarbons.

### BACKGROUND

Processes based on Autothermal Reforming (ATR) is a route to production of synthesis gas. The main elements of an ATR reactor are a burner, a combustion chamber, and a catalyst bed contained within a refractory lined pressure shell. In an ATR reactor, partial combustion of the hydrocarbon feed by sub-stoichiometric amounts of oxygen is followed by steam reforming of the partially combusted hydrocarbon feed stream in a fixed bed of steam reforming catalyst. Steam reforming also takes place to some extent in the combustion chamber due to the high temperature. The steam reforming reaction is accompanied by the water gas shift reaction. Typically, the gas is at or close to equilibrium at the outlet of the reactor with respect to steam reforming and water gas shift reactions. The temperature of the exit gas is typically in the range between 850° and 1100° C. More details of ATR and a full description can be found in the art such as “Studies in Surface Science and Catalysis, Vol. 152,” Synthesis gas production for FT synthesis“; Chapter 4, p. 258-352, 2004”.

It is an object of the invention to provide an alternative configuration of a chemical plant for production of a chemical product.

It is also an object of the invention to provide a system and process for producing synthesis gas by reforming wherein the overall energy consumption is reduced compared to a system with a single fired reforming reactor, such as a tubular steam methane reformer, an autothermal reformer or convective reformer.

It is also an object of the invention to provide a plant and process wherein the capacity of an existing reforming reactor, such as a fired reforming reactor or an autothermal reformer may be increased.

It is also an object of the invention to provide a plant and process allowing high flexibility in the composition of the generated synthesis gas.

It is furthermore an object of the invention to provide a chemical plant and process wherein the overall emission of carbon dioxide and other emissions, such as NOₓ, SOₓ, etc., detrimental to the climate are reduced by minimizing the consumption of hydrocarbons for providing heat for the reforming reactions.

### SUMMARY OF THE INVENTION

In the following, reference is made to embodiments of the invention. However, it should be understood that the invention is not limited to specific described embodiments. Instead, any combination of the following features and elements, whether related to different embodiments or not, is contemplated to implement and practice the invention.

An aspect of the invention relates to chemical plant comprising:
a reforming section arranged to receive a feed gas comprising hydrocarbons and provide a combined synthesis gas stream, wherein said reforming section comprises:
   an electrically heated reforming reactor housing a first catalyst, said electrically heated reforming reactor being arranged for receiving a first part of said feed gas and generating a first synthesis gas stream,
   an autothermal reforming reactor in parallel with said electrically heated reforming reactor, said autothermal reforming reactor housing a second catalyst, said autothermal reforming reactor being arranged for receiving a second part of said feed gas and outputting a second synthesis gas stream,
   wherein said reforming section is arranged to output a combined synthesis gas stream comprising at least part of said first and/or second synthesis gas streams,
an optional post processing unit downstream the reforming section, where said optional post processing unit is arranged to receive the combined synthesis gas stream and provide a post processed synthesis gas stream,
a water separation unit arranged to separate said combined synthesis gas stream or said post processed synthesis gas stream into a water condensate and an intermediate synthesis gas, and
a downstream section arranged to receive the intermediate synthesis gas and to process the intermediate synthesis gas to a chemical product and an off-gas.

In some embodiments all of the first and/or second synthesis gas is output from the reforming section as the combined synthesis gas stream; however, in other embodiments only a part of the first and/or all some of the second synthesis, such as e.g. 20 vol % of the first and/or second synthesis gas stream, is output as the combined synthesis gas stream, whilst other parts thereof are output as synthesis gas for other purposes.

In a case where it is desired to increase the overall synthesis gas production within the reforming section of a chemical plant, where the only reforming reactor is an autothermal reforming reactor, it is an advantage to supplement the autothermal reforming reactor with an electrically heated reforming reactor instead of e.g. a Steam Methane Reformer (SMR) or a gas heated reforming reactor, incl. a heat exchange reformer. This is at least due to:
This combination provides for a lower accumulated generation of carbon dioxide compared to the combination of an autothermal reforming reactor and an SMR, in particular if the electrical power for the electrically heated reforming reactor is from renewable sources,
The overall emission of carbon dioxide and other emissions detrimental to the climate, such as NOₓ or SOₓ, are reduced considerably by minimizing the amount of hydrocarbons used for providing heat for the reforming reactions;
the electrically heated reforming reactor renders it possible to output the first synthesis gas with a higher temperature and/or a higher pressure than what is possible from an SMR, which thereby ensures that the methane content of the first synthesis gas and hence the methane content of the combined synthesis gas may be reduced;
The pressure of the combined synthesis gas can be higher because especially the SMR is confined in maximum pressures in the order of 25 barg, compared to autothermal reforming and electrically heated reforming which both can operate at pressures exceeding 30 barg, more preferably exceeding 40 barg;
The operating conditions of a gas heated reforming reactor are confined to high steam to carbon ratio in order to avoid metal dusting, which is not the case for the electrically heated reforming reactor;
The size of the electrically heated reforming reactor is significantly smaller than an SMR or a gas heated reforming reactor, and therefore makes implementation into an existing plot plan easier;
The H₂/CO ratio of the combined synthesis gas output from the reforming section can be adjusted by controlling the amount of first part of the feed gas to the electrically heated reforming reactor and the amount of the second part of the feed gas to the autothermal reforming reactor, and thereby indirectly controlling of oxygen consumption;
Moreover, the module M of the post processed synthesis gas stream may be tailored. The module M is the stoichiometric ratio (H₂—CO₂)/(CO+CO₂). The module M may be tailored to about 1.8-2.2, more preferably about 2.0 or 2.1, useful in the case where the downstream section comprises a methanol reactor arranged to convert the intermediate synthesis gas to methanol.

In more detail the technical advantages of the plant of the invention may be explained as follows: An ATR typically produces an output gas with a temperature of 1000° C. or more and with a pressure of up to 45 barg. Conventional SMR and gas-heated reformers produce an output gas with a temperature of about 850° C. and a pressure of 25-30 barg. An SMR is typically excluded from operation at higher pressures due to mechanical limitations and a gas-heated reformer is excluded from operation at higher pressures, because the conversion of methane would be unfavorably low at the associated maximum temperature. Overall, this means that the methane conversion will be relatively low in an SMR and in a gas-heated reformer due to the relatively low exit temperature, and when mixed with the output gas from the SMR or gas-heated reformer, the result is an increase of the content of methane in the gas and accordingly in the combined synthesis gas. Also, the pressure limitations of the SMR or the gas-heated reformer means that when the output gas from the ATR and the conventional SMR or gas-heated reformer are to be mixed, it is necessary to reduce the pressure of the output gas from the ATR to the same level as the pressure of the output gas from the conventional SMR or gas-heated reformer. The reduced pressure of the combined synthesis gas means that the requirement of downstream compression of the combined synthesis gas will increase, as many applications of the synthesis gas, such as methanol synthesis (typically above 70 bars), require high pressures. The present invention is based on the recognition that it is possible to produce an output gas from an electrically heated steam methane reformer, which has the same high temperature and pressure as the output gas from the ATR and hence to avoid the said reduction of pressure in the output gas from the ATR and thereby to produce a combined synthesis gas with a reduced content of methane. Thus, it has surprisingly been found that in an electrically heated steam methane reformer it is possible to produce an output gas with a temperature of up to about 1100 or more and a pressure of as high as up to 100 barg.

In addition to the second part of the feed gas input into the autothermal reforming reactor, a stream of oxidant gas is inlet. The stream of oxidant gas comprises oxygen and may be e.g. air or oxygen, or a mixture of more than 90% oxygen with the balance being e.g nitrogen, steam, and/or argon.

It should be noted that the first, second and optional third part of the feed gas comprising hydrocarbons may be a first, second and optional third part of a single feed gas stream comprising hydrocarbons, where the single feed gas stream is split up into streams fed into the first, second and optional third reforming reactors, possibly together with steam. In this case, the composition of the first, second and optional third part of the feed gas is substantial identical. However, additional gasses, such as an oxidant gas and/or steam, may be added to the first, second and optional third part of the feed gas before they are fed into the respective reforming reactors. Even though the first, second and optional third feed gasses may be input individually to the reforming reactors of the reforming section, the term “feed gas” received by the reforming section is meant to denote the total amount of feed gas fed to the reforming reactors. Thus, when the reforming section comprises an electrically heated reforming reactor receiving a first part of the feed gas and an autothermal reforming reactor receiving a second part of the feed gas, the term “feed gas” is meant to denote the total feed gas comprising the first and second parts of the feed gas fed. Similarly, when the reforming section comprises an electrically heated reforming reactor receiving a first part of the feed gas, an autothermal reforming reactor receiving a second part of the feed gas and a gas heated reforming reactor receiving a third part of the feed gas, the term “feed gas” is meant to denote the total feed gas comprising the first, second and third parts of the feed gas fed.

The chemical plant of the invention provides for an increase in the production of the combined synthesis gas of the reforming section. Alternative ways to increase the production of the reforming section would be to combine a fired steam methane reformer and an autothermal reforming reactor or to combine an autothermal reforming reactor with a heat exchange reforming reactor. The combination of an electrically heated reforming reactor and an autothermal reforming reactor is superior to the combination of a fired steam methane reforming reactor and an autothermal reforming reactor since the overall CO₂ emission is reduced and since the temperature and/or the pressure of the combined synthesis gas is higher in the former combination. Moreover, the combination of an electrically heated reforming reactor and an autothermal reforming reactor is superior to the combination of an autothermal reforming reactor and a heat exchange reforming reactor since a heat exchange reforming reactor is confined to operation at high steam to carbon ratios to avoid metal dusting problems.

The chemical plant of the invention provides a concept where synergy is obtained between an electrically heated reforming reactor and the operation of an autothermal reforming reactor. By placing an electrically heated reforming reactor in parallel to an autothermal reforming reactor, the two reforming reactors can collectively use the same preheating and pre-conditioning system or parts thereof. Moreover, by letting a part of the reforming reaction take place within an electrically heated reforming reactor, the import of hydrocarbons to provide heat for the steam reforming reactions is reduced compared to the use of a steam methane reforming reactor in parallel to an autothermal reforming reactor. Thus, the overall consumption of hydrocarbons is minimized for a given output of combined synthesis gas from the reforming section.

Furthermore, by combining an autothermal reforming reactor and an electrically heated reforming reactor the composition of the synthesis gas exiting the reforming section may be controlled. This is in particular useful if the downstream section for example is a methanol synthesis section.

The capacity of an existing chemical plant with an autothermal reforming reactor may be boosted by adding an electrically heated reforming reactor with little, if any, increase in the usage of hydrocarbons for the heating side of the reforming section since the electricity for the electrically heated reforming reactor may be provided from renewable sources, such as wind energy. Moreover, since an electrically heated reforming reactor is a very compact reactor, it may typically be fitted on to the same piece of land as the existing chemical plant.

The downstream section may e.g. be a cold box, a pressure swing adsorption unit, a methanol synthesis section, an ammonia section or a Fischer-Tropsch section. Other downstream sections are also conceivable, such as a downstream section for acetic acid production or DME production.

In a fired tubular steam methane reformer, heat transfer by convection and/or radiation heating can be slow and will often meet large resistance. The temperature at the innermost part of the tubes of the fired tubular steam methane reformer is somewhat lower than the temperature outside the tubes due to the heat transfer rate through the walls of the tube and to the catalyst within the tubes as well due to the endothermic nature of the steam reforming reaction. In the electrically heated reforming reactor, the maximum temperature may be obtained in close vicinity to the first catalyst. Thus, by utilizing electric heating, a high temperature flue gas of the fired steam methane reformer is avoided and less energy is therefore needed in the reforming section of the electrically heated reactor. Moreover, the overall emission of carbon dioxide and other emissions detrimental to the climate, such as NOₓ or SOₓ, are reduced by minimizing the amount of hydrocarbons used for providing heat for the reforming reactions.

Moreover, if the electricity utilized for heating the electrically heated reforming reactor and possibly other units of the chemical plant is provided from renewable energy resources, the overall consumption of hydrocarbons for the chemical plant is minimized and CO₂ emissions accordingly reduced.

Typically, the combined synthesis gas stream from the reforming section contains the first and second synthesis gas streams. Hereby, the further processing of the combined synthesis gas from the reforming section is carried out on all the first and second synthesis gas streams in combination. However, it is conceivable that the combined synthesis gas stream only contains a part of the first and/or the second synthesis gas stream and that the remaining synthesis gas stream is led to other equipment downstream the reforming section. This could e.g. be the case where the chemical plant is arranged to provide one chemical product in the form of a hydrogen gas stream and another chemical product in the form of a CO rich synthesis gas stream.

In this context, the term “feed gas comprising hydrocarbons” is meant to denote a gas with one or more hydrocarbons and possibly other constituents. Thus, typically feed gas comprising hydrocarbons comprises a hydrocarbon gas, such as CH₄ and optionally also higher hydrocarbons in often relatively small amounts, in addition to small amounts of other gasses. Higher hydrocarbons are components with two or more carbon atoms such as ethane and propane. Examples of “hydrocarbon gas” may be natural gas, LPG, town gas, bio-gas, naphtha or a mixture of methane and higher hydrocarbons. Hydrocarbons may also be components with other atoms than carbon and hydrogen such as oxygenates. The term “feed gas comprising hydrocarbons” is meant to denote a feed gas comprising a hydrocarbon gas with one or more hydrocarbons mixed with steam, hydrogen and possibly other constituents, such as carbon monoxide, carbon dioxide, and nitrogen and argon. Typically, the feed gas(ses) let into the reforming section has (have) a predetermined ratio of hydrocarbon gas, steam and hydrogen, and potentially also carbon dioxide. It should be noted, that a feed gas comprising hydrocarbons which is cleaned up, e.g. desulfurized, and/or pre-reformed, is still considered to be a feed gas comprising hydrocarbons.

Moreover, the term “steam reforming” or “steam methane reforming reaction” is meant to denote a reforming reaction according to one or more of the following reactions:

CH₄+H₂O↔CO+3H₂ (i)

CH₄+2H₂O↔CO₂+4H₂ (ii)

CH₄+CO₂↔2CO+2H₂ (iii)

Reactions (i) and (ii) are steam methane reforming reactions, whilst reaction (iii) is the dry methane reforming reaction.

For higher hydrocarbons, viz. CₙHₘ, where n≥2, m≥4, equation (i) is generalized as:

CₙHₘ+nH₂O↔nCO+(n+m/2)H₂ (iv)

where n≥2, m≥4.

Typically, steam reforming is accompanied by the water gas shift reaction (v):

CO+H₂O↔CO₂+H₂ (v)

The terms “steam methane reforming” and “steam methane reforming reaction” is meant to cover the reactions (i) and (ii), the term “steam reforming” is meant to cover the reactions (i), (ii) and (iv), whilst the term “methanation” covers the reverse reaction of reaction (i). In most cases, all of these reactions (i)-(v) are at, or close to, equilibrium at the outlet from the reforming reactor. The term “prereforming” is often used to cover the catalytic conversion of higher hydrocarbons according to reaction (iv). Prereforming is typically accompanied by steam reforming and/or methanation (depending upon the gas composition and operating conditions) and the water gas shift reaction. Prereforming is often carried out in adiabatic reactors but may also take place in heated reactors.

In the case of autothermal reforming, the steam methane reforming is preceded by a reaction zone where combustion and partial combustion of the feedstock takes place.

Moreover, the terms “autothermal reforming” and “autothermal reforming reactions” also cover combustion and partial combustion of the hydrocarbon feedstock according to reaction (vi) and (vii):

CH₄+½O₂↔CO+2H₂ (vi)

CH₄+2O₂↔CO₂+2H₂O (vii)

in addition to the steam methane reforming reactions.

The term “synthesis gas” is meant to denote a gas comprising hydrogen, carbon monoxide and also carbon dioxide and small amounts of other gasses, such as argon, nitrogen, methane, etc.

Typically, the feed gas will have undergone desulfurization to remove sulfur therein and thereby avoid deactivation of the catalysts in the process, prior to being inlet into the reforming section.

In an embodiment, the chemical plant further comprises a gas purification unit and/or a prereforming unit upstream the reforming section. The gas purification unit is e.g. a desulfurization unit, such as a hydrodesulfurization unit.

In the prereformer, the hydrocarbon gas will, together with steam, and potentially also hydrogen and/or other components such as carbon dioxide, undergo prereforming according to reaction (iv) in a temperature range of ca. 350-550° C. to convert higher hydrocarbons as an initial step in the process, normally taking place downstream the desulfurization step. This removes the risk of carbon formation from higher hydrocarbons on catalyst in the subsequent process steps. Optionally, carbon dioxide or other components may also be mixed with the gas leaving the prereforming step to form the feed gas.

In an embodiment, the water separation unit of the chemical plant is a flash separation unit often preceded by suitable temperature reduction equipment. By flash separation is meant a phase separation unit, where a stream is divided into a liquid and gas phase close to or at the thermodynamic phase equilibrium at a given temperature.

In an embodiment, the electrically heated reforming reactor of the chemical plant comprises:
a pressure shell housing an electrical heating unit arranged to heat the first catalyst, where the first catalyst comprises a catalytically active material operable to catalyzing steam reforming of the first feed gas, wherein the pressure shell has a design pressure of between 5 and 45 bar, preferably between 30 and 45 bar,
a heat insulation layer adjacent to at least part of the inside of the pressure shell, and
at least two conductors electrically connected to the electrical heating unit and to an electrical power supply placed outside the pressure shell,
wherein the electrical power supply is dimensioned to heat at least part of the first catalyst to a temperature of at least 800° C., preferably at least 950° C., or even more preferably at least 1050° C. by passing an electrical current through the electrical heating unit.

An important feature of the electrically heated reforming reactor is that the energy is supplied inside the reforming reactor, instead of being supplied from an external heat source via heat conduction, convection and radiation, e.g. through catalyst tubes. In an electrically heated reforming reactor with an electrical heating unit connected to an electrical power supply via conductors, the heat for the steam reforming reaction is provided by resistance heating. The hottest part of the electrically heated reforming reactor will be within the pressure shell of the electrically heated reforming reactor. Preferably, the electrical power supply and the electrical heating unit within the pressure shell are dimensioned so that at least part of the electrical heating unit reaches a temperature of 850° C., preferably 900° C., more preferably 1000° C. or even more preferably 1100° C.

The chemical plant of the invention may advantageously comprise one or more compressors and/or pumps upstream the reforming section. The compressors/pumps are arranged to compress the feed to a pressure of between 5 and 45 bar, preferably between 30 and 45 bar. The constituents of the feed, viz. water/steam, hydrogen and hydrocarbon feed gasses, may be compressed individually and fed individually into the reforming section or to the reforming reactors thereof.

The first catalyst may be a bed of catalyst particles, e.g. pellets, typically in the form of catalytically active material supported on a high area support with electrically conductive structures embedded in the bed of catalyst particles. Alternative, the first catalyst may be catalytically active material supported on a macroscopic structure, such as a monolith.

When the electrically heated reforming reactor comprises a heat insulation layer adjacent to at least part of the inside of the pressure shell, appropriate heat and electrical insulation between the electrical heating unit and the pressure shell is obtained. Typically, the heat insulation layer will be present at the majority of the inside of the pressure shell to provide thermal insulation between the pressure shell and the electrical heating unit/first catalyst; however, passages in the heat insulation layers are needed in order to provide for connection of conductors between the electrical heating unit and the electrical power supply and to provide for inlets/outlets for gasses into/out of the electrically heated reforming reactor.

The presence of heat insulating layer between the pressure shell and the electrical heating unit assists in avoiding excessive heating of the pressure shell and assists in reducing thermal losses to the surroundings of the electrically heated reforming reactor. The temperatures of the electrical heating unit may reach up to about 1300° C., at least at some parts thereof, but by using the heat insulation layer between the electrical heating unit and the pressure shell, the temperature of the pressure shell can be kept at significantly lower temperatures of e.g. 500° C. or even 200° C. This is advantageous since typical construction steel materials are unsuitable for pressure bearing applications at high temperatures, such as above 1000° C. Moreover, a heat insulating layer between the pressure shell and the electrical heating unit assists in control of the electrical current within the reforming reactor, since heat insulation layer is also electrically insulating. The heat insulation layer could be one or more layers of solid material, such as ceramics, inert material, refractory material or a gas barrier or a combination thereof. Thus, it is also conceivable that a purge gas or a confined gas constitutes or forms part of the heat insulation layer.

As the hottest part of the electrically heated reforming reactor during operation is the electrical heating unit, and since a heat insulation layer thermally insulates the pressure shell from the electrically heated reforming reactor, the temperature of the pressure shell can be kept significantly lower than the maximum process temperature. This allows for having a relative low design temperature of the pressure shell of e.g. 700° C. or 500° C. or preferably 300° C. or 200° C. of the pressure shell whilst having maximum process temperatures of 900° C. or even 1100° C. or even up to 1300° C.

Another advantage is that the lower design temperature compared to a fired SMR means that in some cases the thickness of the pressure shell can be decreased, thereby saving costs.

It should be noted that the term “heat insulating material” is meant to denote materials having a thermal conductivity of about 10 W·m⁻¹·K⁻¹ or below. Examples of heat insulating materials are ceramics, refractory material, alumina-based materials, zirconia based materials and similar.

In an embodiment, the electrical heating unit comprises a macroscopic structure of electrically conductive material, where the macroscopic structure supports a ceramic coating and the ceramic coating supports the catalytically active material of the first catalyst. Thus, during operating of the chemical plant, an electrical current is passed through the macroscopic structure and thereby heats the macroscopic structure and the catalytically active material supported thereon. The close proximity between the catalytically active material and the macroscopic structure enables efficient heating of the catalytically active material by solid material heat conduction from the resistance heated macroscopic structure. The amount and composition of the catalytically active material can be tailored to the steam reforming reaction at the given operating conditions. The surface area of the macroscopic structure, the fraction of the macroscopic structure coated with a ceramic coating, the type and structure of the ceramic coating, and the amount and composition of the catalytically active material may be tailored to the steam reforming reaction at the given operating conditions.

The term “electrically conductive” is meant to denote materials with an electrical resistivity in the range from: 10⁻⁴ to 10⁻⁸ Ω·m at 20° C. Thus, materials that are electrically conductive are e.g. metals like copper, silver, aluminum, chromium, iron, nickel, or alloys of metals. Moreover, the term “electrically insulating” is meant to denote materials with an electrical resistivity above 10 Ω·m at 20° C., e.g. in the range from 10⁹ to 10²⁵ Ω·m at 20° C.

As used herein, the term “electrical heating unit comprises a macroscopic catalyst” is not meant to be limited to a reforming reactor with a single macroscopic structure. Instead, the term is meant to cover both a macroscopic structure with ceramic coating and catalytically active material supported thereon as well as an array of such macroscopic structures with ceramic coating and catalytically material supported thereon.

The term “macroscopic structure supporting a ceramic coating” is meant to denote that the macroscopic structure is coated by the ceramic coating at, at least, a part of the surface of the macroscopic structure. Thus, the term does not imply that all the surface of the macroscopic structure is coated by the ceramic coating; in particular, at least the parts of the macroscopic structure which are electrically connected to the conductors and thus to the electrical power supply do not have a coating thereon. The coating is a ceramic material with pores in the structure which allows for supporting the catalytically active material of the first catalyst on and inside the coating and has the same function as a catalytic support. Advantageously, the catalytically active material of the first catalyst comprises catalytically active particles having a size in the range from about 5 nm to about 250 nm.

As used herein, the term “macroscopic structure” is meant to denote a structure which is large enough to be visible with the naked eye, without magnifying devices. The dimensions of the macroscopic structure are typically in the range of centimeters or even meters. Dimensions of the macroscopic structure are advantageously made to correspond at least partly to the inner dimensions of the pressure shell, saving room for the heat insulation layer and conductors.

A ceramic coating, with or without catalytically active material, may be added directly to a metal surface by wash coating. The wash coating of a metal surface is a well-known process; a description is given in e.g. Cybulski, A., and Moulijn, J. A., Structured catalysts and reactors, Marcel Dekker, Inc, New York, 1998, Chapter 3, and references herein. The ceramic coating may be added to the surface of the macroscopic structure and subsequently the catalytically active material may be added; alternatively, the ceramic coat comprising the catalytically active material is added to the macroscopic structure.

Preferably, the macroscopic structure has been manufactured by extrusion of a mixture of powdered metallic particles and a binder to an extruded structure and subsequent sintering of the extruded structure, thereby providing a material with a high geometric surface area per volume. A ceramic coating, which may contain the catalytically material, is provided onto the macroscopic structure before a second sintering in an oxidizing atmosphere, in order to form chemical bonds between the ceramic coating and the macroscopic structure. Alternatively, the catalytically active material may be impregnated onto the ceramic coating after the second sintering. When chemical bonds are formed between the ceramic coating and the macroscopic structure, an especially high heat conductivity between the electrically heated macroscopic structure and the catalytically active material supported by the ceramic coating is possible. Due to close proximity between the heat source, viz. the macroscopic structure, and the catalytically active material, the heat transfer is effective, so that the catalytically active material can be very efficiently heated. A compact reforming reactor in terms of gas processing per reforming reactor volume is thus possible, and therefore the reforming reactor housing the macroscopic structure may be compact. The reforming reactor of the invention does not need a furnace and this reduces the size of the electrically heated reforming reactor considerably.

In another embodiment the macroscopic mixture is manufactured by 3D-printing and/or additive manufacturing.

Preferably, the macroscopic structure comprises Fe, Ni, Cu, Co, Cr, Al, Si or an alloy thereof. Such an alloy may comprise further elements, such as Mn, Y, Zr, C, Co, Mo or combinations thereof. Preferably, the catalytically active material of the first catalyst is particles having a size from 5 nm to 250 nm. The catalytically active material of the first catalyst may e.g. comprise nickel, ruthenium, rhodium, iridium, platinum, cobalt, or a combination thereof. Thus, one possible catalytically active material of the first catalyst is a combination of nickel and rhodium and another combination of nickel and iridium. The ceramic coating may for example be an oxide comprising Al, Zr, Mg, Ce and/or Ca. Exemplary coatings are calcium aluminate or a magnesium aluminum spinel. Such a ceramic coating may comprise further elements, such as La, Y, Ti, K or combinations thereof. Preferably, the conductors are made of different materials than the macroscopic structure. The conductors may for example be of iron, nickel, aluminum, copper, silver, or an alloy thereof. The ceramic coating is an electrically insulating material and will typically have a thickness in the range of around 100 μm, say 10-500 μm. In addition, a sixth catalyst may be placed within the pressure shell and in channels within the macroscopic structure, around the macroscopic structure or upstream and/or upstream the macroscopic structure to support the catalytic function of the macroscopic structure.

In an embodiment, the chemical plant further comprises a control system arranged to control the electrical power supply to ensure that the temperature of the gas exiting the electrically heated reforming reactor lies in a predetermined range and/or to ensure that the conversion of hydrocarbons in the first part of the feed gas lies in a predetermined range and/or to ensure the dry mole concentration of methane lies in a predetermined range and/or to ensure the approach to equilibrium of the steam reforming reaction lies in a predetermined range. Typically, the maximum temperature of the gas within the electrically heated reforming reactor lies between 800° C. and 1000° C., such as between 850° C. and 1000° C., such as at about 950° C., but even higher temperatures are conceivable, e.g. up to 1300° C. The maximum temperature of the first synthesis gas will be achieved close to the most downstream part of the first catalyst as seen in the flow direction of the first part of the feed gas.

The control of the electrical power supply is the control of the electrical output from the power supply. The control of the electrical power supply may e.g. be carried out as a control of the voltage and/or current from the electrical power supply, as a control of whether the electrical power supply is turned on or off or as a combination hereof. The power supplied to the electrical heating unit of the first catalyst can be in the form of alternating current or direct current.

In an embodiment, the chemical plant further comprises a fired heater unit upstream the autothermal reforming reactor (ATR reactor), where the fired heater unit is arranged to preheat the second part of the feed gas, and optionally means for recycling at least part of the off-gas from the downstream section as fuel to the fired heater unit.

By recycling off-gas from the downstream section back to the fired heater unit, it is rendered possible to maximize the use of hydrocarbons in the feed on the process side and minimize the use of hydrocarbons of the fired heating unit. It is possible to balance the chemical plant so that the operation of the fired heating unit is adjusted to being primarily, or even fully, driven by heat supplied by burning a recycled off-gas. This allows for a minimum use of natural gas imported for being burned off for heat in the chemical plant, which in turn allows for an optimal utilization of feed gasses comprising hydrocarbons to the chemical plant. Typically, a relatively small amount of make-up gas comprising hydrocarbon is also fed to the fired heating unit in order to allow control of the duty of the fired heating unit. The term “duty” is in this context understood as the heat input added to or removed from a unit operation in a chemical plant.

In an embodiment, the reforming section furthermore comprises a fired steam methane reforming reactor upstream the autothermal reforming reactor, wherein the fired steam methane reforming reactor comprises one or more tubes housing a third catalyst, wherein the fired steam methane reforming reactor comprises one or more burners for providing heat for the steam methane reforming reaction within the one or more tubes, and wherein the chemical plant comprises means for recycling at least part of the off-gas from the downstream section as fuel to the one or more burners of the fired steam methane reforming reactor, where the fired steam methane reforming reactor is arranged to receive the second part of the feed gas and to provide a partially reformed second feed gas, and wherein the partially reformed second feed gas is led to the autothermal reforming reactor.

A typical fired steam methane reforming reactor has a number of tubes filled with catalyst pellets placed inside a furnace. The tubes are typically 10-13 meters long and will typically have an inner diameter between 80 and 160 mm. Burners placed in the furnace provide the required heat for the reactions by combustion of a fuel gas.

The fuel gas for these fired processes is typically a mix of off-gas(ses) from the process downstream the reformer(s) and import of natural gas or other suitable hydrocarbons.

Since the temperature of the partially reformed second feed gas leaving the fired steam methane reforming reactor may be relatively high, such as 700° C. to 900° C., the second part of the feed gas need not be pre-heated in a separate fired heater unit prior to being led into the autothermal reforming reactor.

In an embodiment, the reforming section of the chemical plant furthermore comprises a gas heated steam methane reforming reactor in parallel to the combination of the electrically heated reforming reactor and the autothermal reforming reactor. The gas heated steam methane reforming reactor comprises a fourth catalyst and is operable to receive a third part of the feed gas and to utilize at least part of the first and/or second synthesis gas streams as heating media in heat exchange within the gas heated steam methane reforming reactor. The gas heated steam methane reforming reactor is arranged for generating a third synthesis gas stream over the fourth catalyst and for outputting the third synthesis gas stream from the reforming section as at least part of the combined synthesis gas. The overall heat efficiency of the chemical plant is increased by the addition of the gas heated steam methane reforming reactor, since the sensitive heat of the first and second synthesis gas streams is used within the gas heated steam methane reforming reactor. Moreover, when the chemical plant includes a gas heated steam methane reforming reactor, the overall output of the chemical plant is increased.

A gas heated steam methane reforming reactor is configured to use a hot gas to supply the heat for the endothermic steam methane reforming reaction by heat exchange, typically over a tube wall. An example of a configuration of a heat exchange reformer has several parallel tubes filled with catalyst which receive the feed gas. In the bottom of the reactor, the product gas from the catalyst filled tubes is mixed with hot synthesis gas from upstream reforming units and the combined synthesis gas carries out heat exchange with the catalyst filled tubes. Other configurations of heat exchange reforming are also conceivable.

Reducing Metal Dusting in Heat Exchange Reforming Reactors

In an embodiment, said reforming section furthermore comprises a gas heated steam methane reforming reactor upstream of said autothermal reforming reactor, wherein said gas heated steam methane reforming reactor comprises a fourth catalyst and being operable to utilize at least part of said second synthesis gas stream as heating media in heat exchange within said gas heated steam methane reforming reactor, said gas heated steam methane reforming reactor being arranged to receive said second part of said feed gas and to provide a partially reformed second feed gas, and wherein the partially reformed second feed gas is led to the autothermal reforming reactor. In a particular embodiment, said gas heated steam methane reforming reactor is further operable to utilize at least part of said first synthesis gas stream as heating media in heat exchange within said gas heated steam methane reforming reactor.

The overall heat efficiency of the chemical plant is increased by the addition of the gas heated steam methane reforming reactor, since the sensitive heat of the first and second synthesis gas streams is used within the gas heated steam methane reforming reactor. Moreover, when the chemical plant includes a gas heated steam methane reforming reactor, the overall output of the chemical plant is increased. A gas heated steam methane reforming reactor in the form of a heat exchange reformer has the inherent technical problem of metal dusting, i.e. corrosion of the metal surfaces of the reactor when exposed to carbon monoxide rich gasses. Metal dusting may be described by the following reaction:

CO+H₂↔C+H₂O (viii)

It has surprisingly been found that when adding an electrically heated reforming reactor in parallel to the combination of a heat exchange reformer and an autothermal reforming reactor, the level of the metal dusting in the heat exchange is strongly reduced. Without being bound by theory, it is believed that this is due to a reduction in the temperature difference between the equilibrium temperature of reaction (viii) and the exit temperature of the second synthesis gas after cooling in the heat exchange reformer. The temperature of the second synthesis gas leaving the het exchange reformer is higher than it would have been if no electrical reformer was included.

Further advantages of the above embodiment include:
The required duty of the heat exchange reformer is reduced, i.e. the size of the reformer is reduced.
The electrically heated reforming reactor is a very compact reactor compared to a fired reactor and a steam reformer hence reducing the plot area of the reactor.
The electrically heated reforming reactor provides a possibility to operate the reactor using solely sustainable power hence minimizing CO₂ emissions.

In a particular embodiment, the first part of the feed gas is less than 25 vol-%, preferably less than 20 vol-%, more preferably less than 15 vol-%, of the total feed gas.

In a particular embodiment, the duty transferred in the electrically heated reforming reactor is less than 40%, preferably less than 30%, and more preferably less than 20% of the total duty transferred in the electrically heated reforming reactor and the gas heated steam methane reforming reactor.

In a particular embodiment, the temperature of the second synthesis gas exiting the gas heated steam methane reforming reactor is higher than 600° C., preferably higher than 650° C., more preferably higher than 700° C.

In a particular embodiment, the difference between the equilibrium temperature of reaction (viii) and the exit temperature of the second synthesis gas after cooling in the heat exchange reformer is less than 250° C., preferably less than 150° C., and more preferably less than 75° C.

The equilibrium temperature of reaction (viii) is found by initially calculating the reaction quotient (Q) of the given gas as:

$$Q = \frac{{y}_{{H}_{2} O}}{{y}_{CO} \cdot {y}_{{H}_{2}}} \cdot {P}^{- 1}$$

Here yⱼ is the molar fraction of compound j, and P is the total pressure in bar. This is used to determine the equilibrium temperature (T_{eq}) at which the given reaction quotient is equal to the equilibrium constant:

Q=K_{COred}(T_{eq})

where K_{COred} is the thermodynamic equilibrium constant of reaction (viii). The equilibrium temperature of reaction (viii) (ΔT_{app,COred}) is then defined as:

ΔT_{app,COred}=T_{eq}−T

where T is the bulk temperature of the gas.

In a particular embodiment, the feed gas is subjected to desulfurization and adiabatic prereforming before divided into the first and second parts of the feed gas. In a particular embodiment of the invention, steam is added to the first part of the feed gas. In a particular embodiment of the invention, steam is added to the second part of the feed gas. In a particular embodiment of the invention, steam is added to both the first and the second part of the feed gas. In a particular embodiment of the invention, the first and second part of the feed gas have identical compositions. In a particular embodiment of the invention, the first and second part of the feed gas have different compositions.

In a particular embodiment, the process of the present invention relates to a process wherein said reforming section furthermore comprises a gas heated steam methane reforming reactor upstream of said autothermal reforming reactor, wherein said gas heated steam methane reforming reactor comprises a fourth catalyst, said process further comprising the steps of:
inletting said second part of the feed gas into said gas heated steam methane reforming reactor, and carrying out steam methane reforming within said fired reforming reactor to provide a partially reformed second feed gas,
providing said partially reformed second feed gas to said autothermal reforming reactor, and
utilizing at least part of said first and/or second synthesis gas streams as heating media in heat exchange within said gas heated steam methane reforming reactor.

A particular embodiment of the process of the preceding paragraph comprises the further step of:

utilizing at least part of said first synthesis gas stream as heating media in heat exchange within said gas heated steam methane reforming reactor.

### Further Embodiments

In an embodiment, the post processing unit is a post conversion unit having an inlet for allowing addition of heated CO₂ to the combined synthesis gas upstream the post conversion unit. The post processing unit houses a fifth catalyst active for catalyzing steam methane reforming, methanation and reverse water gas shift reactions. The post conversion unit is e.g. an adiabatic post conversion unit or a gas heat exchange reactor.

The post processed synthesis gas stream is a synthesis gas stream with an H₂/CO ratio lower than the H₂/CO ratio of the combined synthesis gas. By adding heated CO₂ and carrying out steam methane reforming, methanation and reverse water gas shift reactions in a separate reactor downstream the reforming section, the CO production of the process may be increased and/or the H₂/CO ratio may be tailored. The H₂/CO ratio of the post processed synthesis gas stream is e.g. lower than 1.8, lower than 1.5 or even lower than 1.0. The temperature of the heated CO₂ added may be e.g. a temperature of about 300° C., 400° C. or even of about 500° C. or above.

In an embodiment, the post processing unit is a water gas shift unit arranged to carry out the water gas shift reaction. In this embodiment, the intermediate synthesis gas, viz. the post processed synthesis gas, is a water gas shifted synthesis gas stream, such as a hydrogen rich synthesis gas or a hydrogen gas stream. The water gas shift unit may be a single water gas shift unit, such as a medium temperature water gas shift unit, or a combination of two or more water gas shift units, e.g. a high temperature water gas shift unit and a low temperature water gas shift unit.

In an embodiment, the downstream section comprises gas separation unit(s) arranged to separate a stream of substantially pure CO₂, H₂, and/or CO from the synthesis gas inlet to the downstream section, thereby providing a refined synthesis gas.

Here, the term “refined synthesis gas” is meant to denote a synthesis gas obtained from the intermediate synthesis gas after selective gas separation of either CO, CO₂ or H₂ or of selective gas separation CO₂ as well as CO or H₂. The gas separation unit comprises one or more of the following units: a CO₂ removal unit, a pressure swing adsorption unit, a membrane, and/or a cryogenic separation unit. By CO₂ removal is meant a unit utilizing a process, such as chemical absorption, for removing CO₂ from the process gas. In chemical absorption, the CO₂ containing gas is passed over a solvent which reacts with CO₂ and in this way binds it. The majority of the chemical solvents are amines, classified as primary amines as monoethanolamine (MEA) and digylcolamine (DGA), secondary amines as diethanolamine (DEA) and diisopropanolamine (DIPA), or tertiary amines as triethanolamine (TEA) and methyldiethanolamine (MDEA), but also ammonia and liquid alkali carbonates as K₂CO₃ and NaCO₃ can be used. By swing adsorption, a unit for adsorbing selected compounds is meant. In this type of equipment, a dynamic equilibrium between adsorption and desorption of gas molecules over an adsorption material is established. The adsorption of the gas molecules can be caused by steric, kinetic, or equilibrium effects. The exact mechanism will be determined by the used adsorbent and the equilibrium saturation will be dependent on temperature and pressure. Typically, the adsorbent material is treated in the mixed gas until near saturation of the heaviest compounds and will subsequently need regeneration. The regeneration can be done by changing pressure or temperature. In practice, this means that a process with at least two units is used, saturating the adsorbent at high pressure or low temperature initially in one unit, and then switching unit, now desorbing the adsorbed molecules from the same unit by decreasing the pressure or increasing the temperature. When the unit operates with changing pressures, it is called a pressure swing adsorption unit, and when the unit operates with changing temperature, it is called a temperature swing adsorption unit. Pressure swing adsorption can generate a hydrogen purity of 99.9% or above. By membrane is meant separation over an at least partly solid barrier, such as a polymer, where the transport of individual gas species takes place at different rates defined by their permeability. This allows for up-concentration, or dilution, of a component in the retentate of the membrane. By cryogenic separation is meant a process utilizing the phase change of different species in the gas to separate individual components from a gas mixture by controlling the temperature, typically taking place below −150° C. It should be noted that the gas separation unit potentially also provides a byproduct, stream, such as a CO₂ stream from a CO₂ removal operation.

In an embodiment the downstream section comprises an ammonia reactor to convert the intermediate synthesis gas to ammonia. In another embodiment, the downstream section comprises a methanol reactor to convert the intermediate synthesis gas to methanol. In yet another embodiment, the downstream section comprises a Fischer-Tropsch reactor to convert the intermediate synthesis gas to a mixture of higher hydrocarbons.

In an embodiment, the first, second, third, fourth fifth, and/or sixth catalysts are catalysts suitable for the steam reforming reaction, the prereforming reaction, methanation and/or the water gas shift reaction. Examples of relevant such catalysts are Ni/MgAl₂O₄, Ni/CaAl₂O₄, Ni/Al₂O₃, Fe₂O₃/Cr₂O₃/MgO, and Cu/Zn/Al₂O₃. In an embodiment, the first, second, third, fourth fifth, and/or sixth catalyst is a steam reforming catalyst. Examples of steam reforming catalysts are Ni/MgAl₂O₄, Ni/Al₂O₃, Ni/CaAl₂O₄, Ru/MgAl₂O₄, Rh/MgAl₂O₄, Ir/MgAl₂O₄, Mo₂C, Wo₂C, CeO₂, a noble metal on an Al₂O₃ carrier, but other catalysts suitable for reforming are also conceivable.

Another aspect of the invention relates to a process for producing a chemical product from a feed gas comprising hydrocarbons, in a chemical plant comprising a reforming section. The reforming section comprises an electrically heated reforming reactor housing a first catalyst, and an autothermal reforming reactor in parallel with the electrically heated reforming reactor. The autothermal reforming reactor houses a second catalyst. The process comprises the steps of:
inletting a first part of the feed gas to the electrically heated reforming reactor and carrying out steam methane reforming to provide a first synthesis gas stream,
inletting a second part of the feed gas to the autothermal reforming reactor, and carrying out reforming to provide a second synthesis gas stream,
outputting a combined synthesis gas stream comprising at least part of the first and/or second synthesis gas streams from the reforming section,
optionally, in a post processing unit downstream the electrically heated reforming reactor and the autothermal reforming reactor, post processing the combined synthesis gas stream to provide a post processed synthesis gas stream,
separating the combined synthesis gas stream or the post processed synthesis gas stream into a water condensate and an intermediate synthesis gas in a water separation unit downstream the post processing unit, and
providing the intermediate synthesis gas to a downstream section arranged to receive the intermediate synthesis gas and to process the intermediate synthesis gas to a chemical product and an off-gas.

Advantages of the process and embodiments thereof correspond to the advantages of the chemical plant and embodiments thereof and will therefore not be described in further detail here.

However, it should be noted that the first, second and optional third part of the feed gas comprising hydrocarbons may be a first, second and optional third part of a single feed gas stream comprising hydrocarbon, where the single feed gas stream is split up into streams fed into the first, second and optional third reforming reactors, possibly together with steam. In this case, the composition of the first, second and optional third part of the feed gas is substantial identical. However, additional gasses, such as an oxidant gas and/or steam, may be added to the first, second and optional third part of the feed gas before they are fed into the respective reforming reactors.

In an embodiment, the first part of the feed gas is about 5-20 vol % of the feed gas. In the case, where the reforming section comprises an electrically heated reforming reactor and a fired reforming reactor, and no further reactors, the first part of the feed gas to the electrically heated reforming reactor is advantageously about 10-20 vol %, e.g. about 15 vol %, of the feed gas and the second part of the feed gas to the autothermal reforming reactor is thus about 80-90 vol %, e.g. about 85 vol %, of the feed gas.

In an embodiment, where the reforming section comprises a gas heated steam methane reforming reactor, the first part of the feed gas is about 5-10 vol % of the feed gas, the second part of the feed gas is about 80-90 vol % of the feed gas, and the third part of the feed gas is about 5-10 vol % of the feed gas.

Moreover, it should be noted that the order in which the steps of the process are written are not necessarily the order in which the process steps take place, in that two or more steps may take place simultaneously, or the order may be different that indicated above.

### SHORT DESCRIPTION OF THE FIGURES

FIG. 1 shows a chemical plant according to an embodiment of the invention, where the reforming section comprises an autothermal reforming reactor and an electrically heated reforming reactor in parallel;

FIG. 2 shows a chemical gas plant according to an embodiment of the invention, where the reforming section also comprises a fired steam methane reforming reactor upstream the autothermal reforming reactor; and

FIG. 3 shows a chemical plant according to an embodiment of the invention, where the reforming section comprises four reforming reactors.

FIG. 4 shows a chemical gas plant according to an embodiment of the invention, where the reforming section also comprises a gas heated steam methane reforming reactor upstream the autothermal reforming reactor.

### DETAILED DESCRIPTION OF THE FIGURES

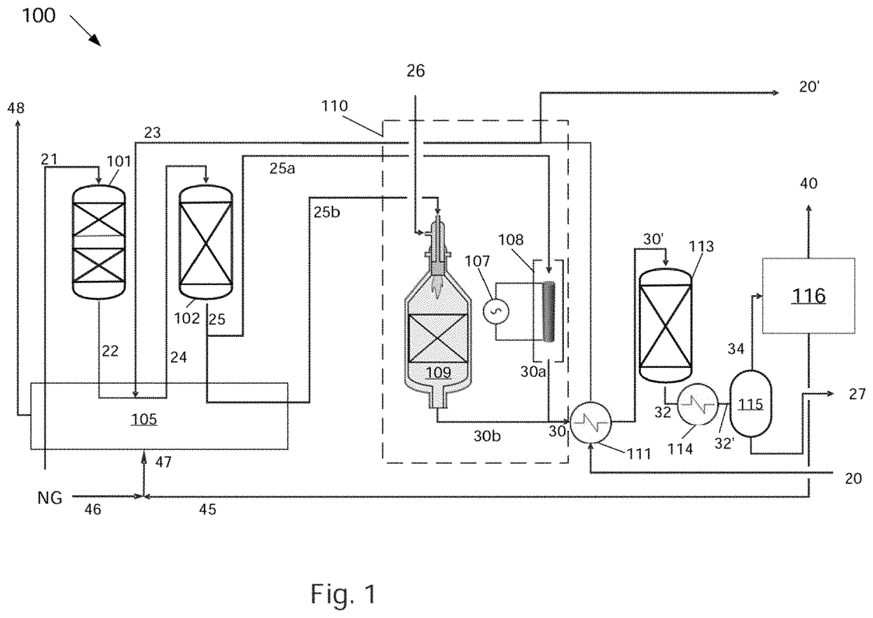

FIG. 1 shows a chemical plant 100 according to an embodiment of the invention. The chemical plant 100 comprises a reforming section 110 with an autothermal reforming reactor 109 and an electrically heated reforming reactor 108 in parallel.

The electrically heated reforming reactor 108 houses a first catalyst and the autothermal reforming reactor 109 houses a second catalyst. The electrically heated reforming reactor 108 is heated by means of an electrical power supply 107.

The electrically heated reforming reactor 108 and autothermal reforming reactor 109 are arranged in parallel. The electrically heated reforming reactor 108 is heated by means of an electrical power supply 107. The electrically heated reforming reactor 108 and autothermal reforming reactor 109 are arranged to receiving a first part 25a and a second part 25b of a feed gas 25 and to generate a first and second synthesis gas 30a, 30b, respectively.

During operation of the chemical plant 100, a feed gas 21 comprising hydrocarbons undergoes feed purification in a desulfurization unit 101 and becomes a desulfurized gas 22. The feed gas 21 comprising hydrocarbons is e.g. natural gas or town gas. The desulfurized gas 22 is preheated in a fired heating unit 105 and steam 23 is added to the desulfurized gas 22, resulting in a gas stream 24. The gas stream 24 is led to a prereforming unit 102 housing steam reforming catalyst. Typically, the prereforming unit 102 is an adiabatic prereforming unit, wherein higher hydrocarbons are reacted so that the prereformed gas 25 exiting the prereforming unit 102 contains no or very small amounts of higher hydrocarbons. The prereformed gas 25 is divided into a first part 25a of the feed gas which is led to the electrically heated reforming reactor 108 and a second part 25b of the feed gas which is led to the autothermal reforming reactor 109. Additional steam may be added to the first part 25a of the feed gas (not shown in FIG. 1). The first catalyst in the electrically heated reforming reactor 108 is a steam methane reforming catalyst arranged to catalyze the steam methane reforming reaction in the electrically heated reforming reactor 108. The autothermal reforming reactor 109 also comprises a steam methane reforming catalyst arranged to carry out steam methane reforming reaction. Air or oxygen 26 is also added to the autothermal reforming reactor 26 in order to carry out partial combustion of the second part of the feed gas 25b upstream the second catalyst within the autothermal reforming reactor 109. A first and second synthesis gas stream 30a, 30b exit the electrically heated reforming reactor 108 and the autothermal reforming reactor 109, respectively, and are combined to a combined synthesis gas stream 30 exiting the reforming section 110. The combined synthesis gas stream 30 is cooled in a heat exchanger 111 to a cooled combined synthesis gas stream 30′. The cooled combined synthesis gas stream 30′ enters a post processing unit 112, viz. a water gas shift unit, and a water gas shifted synthesis gas 32 exits the water gas shift unit 112. The water gas shifted synthesis gas 32 is cooled in a second heat exchanger 113 to a cooled water gas shifted synthesis gas 32′, which enters the water separation unit 115, such as e.g. a flash separation unit 115 arranged to separate the cooled water gas shifted synthesis gas 32′ into a condensate 27 and an intermediate synthesis gas 34. The intermediate synthesis gas 34 is a dry synthesis gas and enters the downstream section 116 arranged to process the intermediate synthesis gas 34 to a chemical product 40 and an off-gas 45. The downstream section 116 comprises e.g. an ammonia reactor to convert the intermediate synthesis gas 34 to ammonia, a methanol reactor to convert the intermediate synthesis 34 gas to methanol, or a Fischer-Tropsch reactor to convert the intermediate synthesis gas 34 to a mixture of higher hydrocarbons.

The off-gas 45 from the downstream section 116 is recycled as fuel to one or more burners of the fired heating unit 105. The off-gas 45 is combined with a small amount of natural gas 46 to form the fuel gas 47 sent to the one or more burners of the fired heating unit 105. The fired heating unit is arranged to provide heat for preheating the feed gas 21, the desulfurized feed gas 22, and the first and/or second part 25a, 25b of the feed gas 25. In FIG. 1, only the second part 25b of the feed gas 25 is heated in the fired heating unit 105 prior to entering into the autothermal reforming reactor 109. However, it is also conceivable that the first part 25a of the feed gas 25 is preheated in the fired heating unit 105.

A heat exchange fluid 20, such as water, is used for heat exchange in the heat exchanger 111 and a heated heat exchange fluid, such as steam, is exported as stream 20′. A part of the steam is used as addition of steam 23 to the desulfurized gas 22.

It should be noted, that the chemical plant 100 typically comprises further equipment, such as compressors, heat exchangers etc.; however, such further equipment is not shown in FIG. 1.

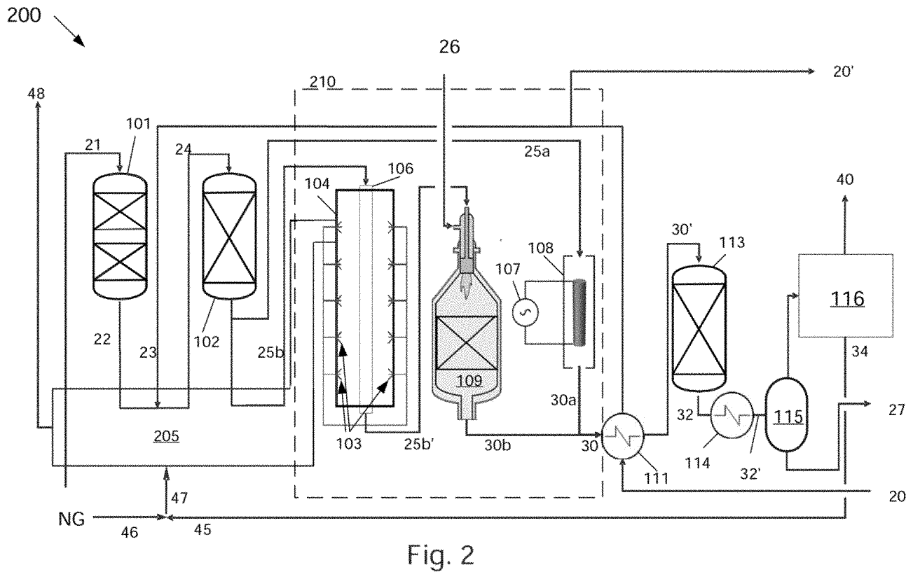

FIG. 2 shows a chemical gas plant 200 according to an embodiment of the invention, where the reforming section 210 also comprises a fired steam methane reforming reactor 104 upstream the autothermal reforming reactor 109.

The chemical plant 200 comprises a reforming section 210 with an electrically heated reforming reactor 208 housing a first catalyst, an autothermal reforming reactor 109 housing a second catalyst and a fired steam methane reforming reactor 104 housing a third catalyst. The fired reforming reactor 104 is a side fired tubular steam methane reforming reactor 104. Thus, the side fired tubular steam methane reforming reactor 104 comprises a number of tubes 106 housing the third catalyst and a number of burners 103 arranged to heat the tubes 106. For the sake of clarity, only one tube 106 is shown in FIG. 2. Fuel is fed to the burners 103 and is burned to provide the heat for the tubes 106. Hot flue gas from the burners 103 is directed to a preheating section 205 of the steam methane reforming reactor 104 and is used for preheating feed gas and steam. The electrically heated reforming reactor 108 is arranged in parallel to the combination of the fired steam methane reforming reactor 104 and the autothermal reforming reactor 109. The electrically heated reforming reactor 108 is heated by means of an electrical power supply 107.

The electrically heated reforming reactor 108 and side fired steam reforming reactor 104 are arranged to receive a first and second feed gas 25a, 25b, respectively, and to generate a first synthesis gas 30a and a pre-reformed feed gas 25b. The pre-reformed feed gas 25b exits the fired reforming reactor at a temperature of between 700° C. and 900° C. and therefore needs no further preheating prior to entering the autothermal reforming reactor 109. A stream 26 of air or oxygen is added to the autothermal reforming reactor 109. The autothermal reforming reactor 109 outputs a second synthesis gas 30b.

During operation of the chemical plant 200, a feed gas 21 comprising hydrocarbons undergoes feed purification in a desulfurization unit 101 and becomes a desulfurized gas 22. The feed gas 21 comprising hydrocarbons is e.g. natural gas or town gas. The desulfurized gas 22 is preheated in the preheating section 205 of the steam methane reformer 104 and steam 23 is added, resulting in a gas stream 24. The gas stream 24 is led to a prereforming unit 102 housing steam reforming catalyst. Typically, the prereforming unit 102 is an adiabatic prereforming unit, wherein higher hydrocarbons are reacted so that the prereformed gas 25 exiting the prereformer contains no or very small amounts of higher hydrocarbons. The prereformed gas 25 is divided into a first part 25a of the feed gas which is led to the electrically heated reforming reactor 208, and a second part 25b of the feed gas which is led to the steam methane reformer 104. The first catalyst in the electrically heated reforming reactor 108, the second catalyst in the autothermal reforming reactor 109 and the third catalyst in the steam methane reformer 104 are steam methane reforming catalysts arranged to catalyze the steam methane reforming reaction in the electrically heated reforming reactor 108, the steam methane reformer 104 and autothermal heated reforming reactor 109.

The electrically heated reforming reactor 108 generates a first synthesis gas 30a, and the steam methane reformer 104 generates a partially reformed synthesis gas 25b and the autothermal reforming reactor 109 provides a second synthesis gas 30b. The first and second synthesis gas 30a, 30b are combined to a synthesis gas stream 30 which is outlet from the reforming section 210 as a combined gas synthesis stream 30.

The combined synthesis gas stream 30 is cooled in a heat exchanger 111 to a cooled combined synthesis gas stream 30′. The cooled combined synthesis gas stream 30′ enters a post processing unit 112, viz. a water gas shift unit, and a water gas shifted synthesis gas 32 exits the water gas shift unit 212. The water gas shifted synthesis gas 32 is cooled in a second heat exchanger 113 to a cooled water gas shifted synthesis gas 32′, which enters the water separation unit 114, e.g. a flash separation unit 115. The cooled water gas shifted synthesis gas 32′ is separated into a condensate 27 and an intermediate synthesis gas 34. The intermediate synthesis gas 34 is a dry synthesis gas which is led to the downstream section 116 arranged to process the intermediate synthesis gas 34 to a chemical product 40 and an off-gas 45.

The downstream section 116 comprises e.g. an ammonia reactor to convert the intermediate synthesis gas 34 to ammonia, a methanol reactor to convert the intermediate synthesis gas 34 to methanol, or a Fischer-Tropsch reactor to convert the intermediate synthesis gas 34 to a mixture of higher hydrocarbons.

An off-gas 45 from the downstream section 116 is recycled as fuel to the burners 103 of the steam methane reformer 104. The off-gas 45 is combined with a small amount of natural gas 46 to form the fuel gas 47 sent to the burners 103 of the steam methane reformer 104. The fuel gas 47 is burnt off in the burners 103, thus heating the tubes 106 with third catalyst. In the preheating section 205, the flue gas from the burners 103 provides heat for preheating the feed gasses and exits as flue gas 48 from the preheating section 205. A heat exchange fluid 20, such as water, is used for heat exchange in the heat exchanger 211 and a heated heat exchange fluid, such as steam, is exported as stream 20′. A part of the steam is used as addition of steam 23 to the pre-sulfurized gas 22.

It should be noted, that the chemical plant 200 typically comprises further equipment, such as compressors, heat exchangers etc.; however, such further equipment is not shown in FIG. 2.

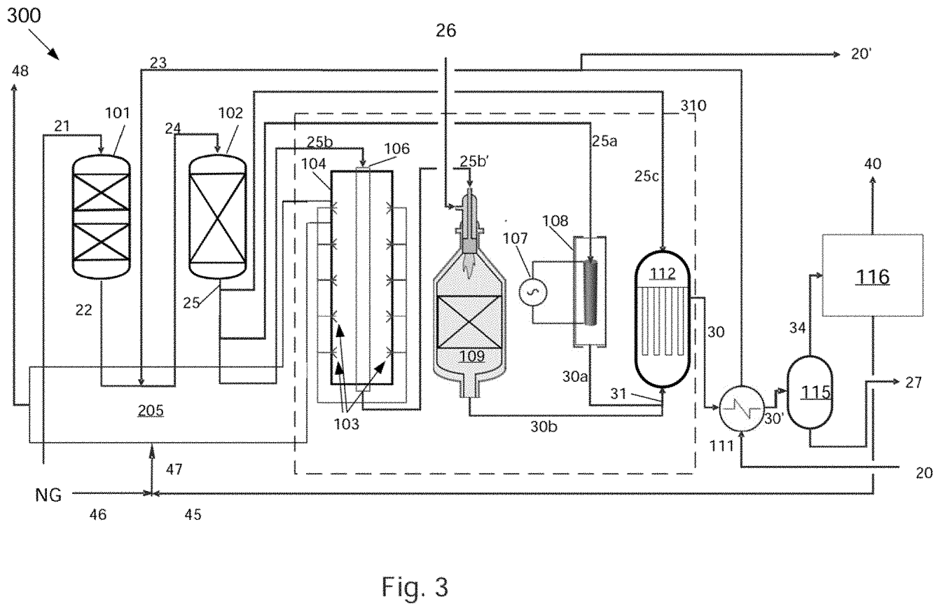

FIG. 3 shows a chemical plant 300 according to an embodiment of the invention, where the reforming section comprises four reforming reactors, namely an electrically heated reforming reactor 108 housing a first catalyst in parallel with the combination of a fired steam reforming reactor 104 housing a third catalyst and an autothermal reactor 109, housing a second catalyst, in addition to a gas heated reactor 112 housing a fourth catalyst.

The fired steam reforming reactor 104 is a side fired, tubular steam methane reforming reactor 104 comprising a number of tubes 106 housing the third catalyst and a number of burners 103 arranged to heat the tubes 106. For the sake of clarity, only one tube is shown in FIG. 3. Fuel is fed to the burners 103 and is burned to provide the heat for the tubes 106. Hot flue gas from the burners 103 is directed to a preheating section 205 of the steam methane reforming reactor 104 and is used for preheating feed gas and steam. The electrically heated reforming reactor 108 is arranged in parallel to the combination of an upstream fired steam reforming reactor 104 and the autothermal reforming reactor 109. The electrically heated reforming reactor 108 is heated by means of an electrical power supply 107.

A first part 25a of the feed gas 25 comprising hydrocarbons is led to the electrically heated reforming reactor 108 and a second part 25b of the feed gas 25 comprising hydrocarbons is led to the side fired steam reforming reactor 104. In the side fired steam reforming reactor 104 the second part 25b of the feed gas 25 is partially reformed to a partially reformed second feed gas 25b, which is fed to the autothermal reforming reactor 109 together with a stream of oxidant gas 26, such as oxygen or air.

During operation of the chemical plant 300, a feed gas 21 comprising hydrocarbons undergoes feed purification in a desulfurization unit 101 and becomes a desulfurized gas 22. The feed gas 21 comprising hydrocarbons is e.g. natural gas or town gas. The desulfurized gas 22 is preheated in the preheating section 205 of the steam methane reforming reactor 104 and steam 23 is added, resulting in a gas stream 24. The gas stream 24 is led to a prereforming unit 102 housing steam reforming catalyst. Typically, the prereforming unit 102 is an adiabatic prereforming unit, wherein higher hydrocarbons are reacted so that the prereformed gas 25 exiting the prereformer contains no or very small amounts of higher hydrocarbons. The prereformed gas 25 is divided into a first part 25a of the feed gas, which is led to the electrically heated reforming reactor 108, a second part 25b of the feed gas which is led to the fired steam methane reforming reactor 104 and a third part 25c of the feed gas which is led to the gas heated steam methane reforming reactor 112.

The first catalyst in the electrically heated reforming reactor 308, the second catalyst in the autothermal reformer 109, the third catalyst in the steam methane reforming reactor 104 and the fourth catalyst in the gas heated steam methane reforming reactor 112 are steam methane reforming catalysts arranged to catalyze the steam methane reforming reaction in the electrically heated reforming reactor 108, the autothermal reformer 109, the steam methane reforming reactor 104 and the gas heated steam methane reforming reactor 112.

The first, second and third part 25a, 25b, 25c, of the feed gas 25, respectively, undergo steam methane reforming in the electrically heated reforming reactor 108, the steam methane reforming reactor 104, the autothermal reforming reactor 109 and the gas heated steam methane reforming reactor 106, respectively. The electrically heated reforming reactor 108 generates a first synthesis gas 30a, whilst the steam methane reforming reactor 104 generates a partially reformed second feed gas 25b′ which is further reformed in the autothermal reforming reactor 109 to provide a second synthesis gas 30b. The first and second synthesis gas 30a, 30b are combined to a synthesis gas stream 31 which is inlet to the gas heated steam methane reforming reactor 112 in order to provide heat for the steam methane reforming reaction of the third part 25c of the feed gas entering the gas heated steam methane reforming reactor 112 from another side.

A synthesis gas steam 30 is outlet from the gas heated steam methane reforming reactor 112 and thereby from the reforming section 310 as a combined gas synthesis stream 30. The combined synthesis gas stream 30 is cooled in a heat exchanger 113 to a cooled combined synthesis gas stream 30′.

The cooled combined synthesis gas stream 30′ enters a water separation unit 114, such as a flash separation unit 115 arranged to separate the cooled combined synthesis gas 30′ into a condensate 27 and an intermediate synthesis gas 34 in the form of a dry synthesis gas. The dry synthesis gas 34 enters the downstream section 116 arranged to process the dry synthesis gas 34 gas to a chemical product 40 and an off-gas 45. The downstream section 116 comprises e.g. an ammonia reactor to convert the intermediate synthesis gas 34 to ammonia, a methanol reactor to convert the intermediate synthesis gas 34 to methanol, or a Fischer-Tropsch reactor to convert the intermediate synthesis gas 34 to a mixture of higher hydrocarbons.

The off-gas 45 from the downstream section 116 is recycled as fuel to the burners 103 of the fired steam methane reforming reactor 104. The off-gas 45 is combined with a small amount of natural gas 46 to form the fuel gas 47 sent to the burners 103 of the steam methane reforming reactor 104. The fuel gas 47 is burnt off in the burners 103, thus heating the tubes 106 with third catalyst. In the preheating section 305, the flue gas from the burners 303 provides heat for preheating the feed gasses and exits as flue gas 48 from the preheating section 305. A heat exchange fluid 20, such as water, is used for heat exchange in the heat exchanger 113 and a heated heat exchange fluid, such as steam, is exported as stream 20′.

It should be noted, that the chemical plant 300 typically comprises further equipment, such as compressors, heat exchangers etc.; however, such further equipment is not shown in FIG. 3.

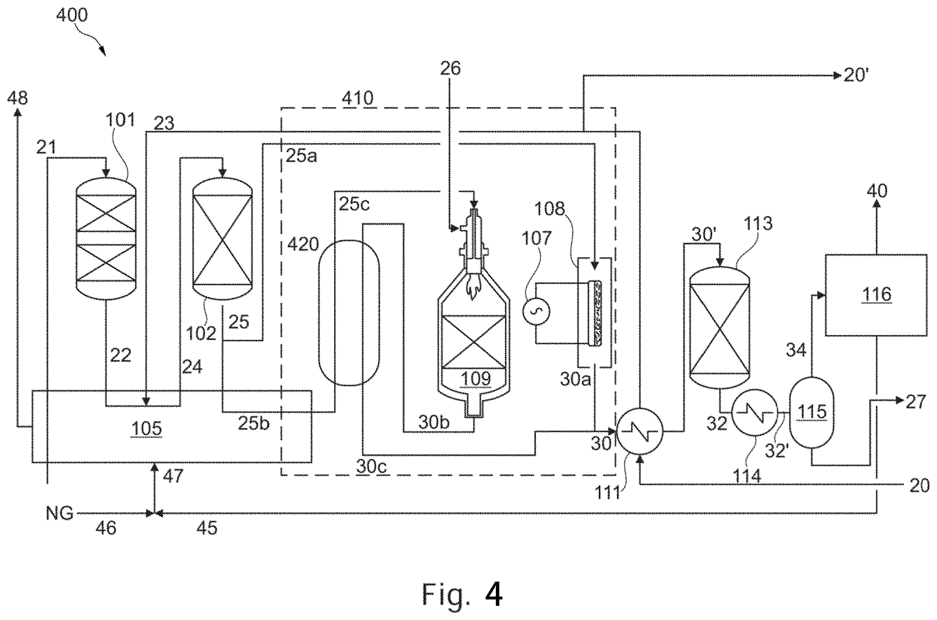

FIG. 4 shows a chemical gas plant 400 according to an embodiment of the invention, where the reforming section 410 also comprises a gas steam methane reforming reactor 420 upstream the autothermal reforming reactor 109.

The second part of the feed gas 25b is heated and prereformed in the gas steam methane reforming reactor 420 to provide a partially reformed second feed gas 25c, and the partially reformed second feed gas 25c is led to the autothermal reforming reactor 109. The second synthesis gas 30b is utilized as heating media in heat exchange within said gas heated steam methane reforming reactor 420 to heat the second part of the feed gas 25b thereby providing a partially cooled second synthesis gas 30c. The partially cooled second synthesis gas 30c is combined with the first synthesis gas 30a to form a combined synthesis gas 30 exiting the reforming section 410.

### EXAMPLE 1

Table 1 shows an example of how an ATR and an electric reformer is integrated for production of a combined synthesis gas. Firstly, by coupling the electric reformer in parallel to the ATR, the production capacity of synthesis gas is increased without additional requirements for oxygen. Secondly, the module of the synthesis gas can be changed, as the H₂/CO ratio out of the ATR is 2.3, which is increased to 2.6 in the combined synthesis gas in the given case.

TABLE 1

| | | | | Stream 25a to | Synthesis gas 30 a | |
|---|---|---|---|---|---|---|
| | | | Second Synthesis | Electrically | from electrically | Combined |
| | Stream 25b | Oxygen 26 | gas 30b from ATR | heated Reform- | heated reforming | synthesis gas |
| | to ATR 109 | to ATR 109 | 109 | ing reactor 108 | reactor 108 | 30 |
| T [° C.] | 625 | 240 | 1050 | 420 | 950 | 1015 |
| P [kg/cm²g] | 39.5 | 39.7 | 38 | 40 | 39.5 | 38 |
| CH₄ | 25027 | 0 | 1291 | 10726 | 2702 | 3993 |
| [Nm³/h] | | | | | | |
| CO | 830 | 0 | 21508 | 356 | 6908 | 28416 |
| [Nm³/h] | | | | | | |
| CO₂ | 616 | 0 | 3675 | 264 | 1736 | 5410 |
| [Nm³/h] | | | | | | |
| H₂ [Nm³/h] | 1527 | 0 | 48482 | 654 | 74680 | 74680 |
| N₂ [Nm³/h] | 0 | 279 | 279 | 0 | 0 | 279 |
| O₂ [Nm³/h] | 0 | 13655 | 0 | 0 | 0 | 0 |
| H₂O | 15016 | 132 | 15663 | 19306 | 9811 | 25474 |
| [Nm³/h] | | | | | | |
| H₂/CO | | | 2.3 | | 10.8 | 2.6 |

1. A chemical plant comprising:
a reforming section arranged to receive a feed gas comprising hydrocarbons and provide a combined synthesis gas stream, wherein said reforming section comprises:
an electrically heated reforming reactor housing a first catalyst, said electrically heated reforming reactor being arranged for receiving a first part of said feed gas and generating a first synthesis gas stream,
an autothermal reforming reactor in parallel with said electrically heated reforming reactor, said autothermal reforming reactor housing a second catalyst, said autothermal reforming reactor being arranged for receiving a second part of said feed gas and outputting a second synthesis gas stream,
wherein said reforming section is arranged to output a combined synthesis gas stream comprising at least part of said first and/or second synthesis gas streams,
an optional post processing unit downstream the reforming section, where said optional post processing unit is arranged to receive the combined synthesis gas stream and provide a post processed synthesis gas stream,
a water separation unit arranged to separate said combined synthesis gas stream or said post processed synthesis gas stream into a water condensate and an intermediate synthesis gas, and
a downstream section arranged to receive the intermediate synthesis gas and to process the intermediate synthesis gas to a chemical product and an off-gas.

2. The chemical plant according to claim 1, wherein said electrically heated reforming reactor comprises:
a pressure shell housing an electrical heating unit arranged to heat said first catalyst, where said first catalyst comprises catalytically active material operable to catalyzing steam reforming of said first part of said feed gas, wherein said pressure shell has a design pressure of between 5 and 45 bar,
a heat insulation layer adjacent to at least part of the inside of said pressure shell, and
at least two conductors electrically connected to said electrical heating unit and to an electrical power supply placed outside said pressure shell,
wherein said electrical power supply is dimensioned to heat at least part of said first catalyst to a temperature of at least 800° C. by passing an electrical current through said electrical heating unit.

3. The chemical plant according to claim 2, wherein said electrical heating unit comprises a macroscopic structure of electrically conductive material, where said macroscopic structure supports a ceramic coating and said ceramic coating supports said catalytically active material.

4. The chemical plant according to claim 1, further comprising:
a fired heater unit upstream said autothermal reforming reactor, the fired heater unit being arranged to preheat said second part of said feed gas, and
means for recycling at least part of said off-gas from said downstream section as fuel to the fired heater unit.

5. The chemical plant according to claim 1, wherein said reforming section furthermore comprises a fired steam methane reforming reactor upstream said autothermal reforming reactor, wherein said fired steam methane reforming reactor comprises one or more tubes housing a third catalyst, wherein said fired steam methane reforming reactor comprises one or more burners for providing heat for the steam methane reforming reaction within said one or more tubes, and wherein said chemical plant comprises means for recycling at least part of said off-gas from said downstream section as fuel to the one or more burners of the fired steam methane reforming reactor, where the fired steam methane reforming reactor is arranged to receive said second part of said feed gas and to provide a partially reformed second feed gas, and wherein the partially reformed second feed gas is led to the autothermal reforming reactor.

6. The chemical plant according to claim 1, wherein said reforming section furthermore comprises a gas heated steam methane reforming reactor in parallel to the combination of said electrically heated steam methane reforming reactor and the autothermal reforming reactor, wherein said gas heated steam methane reforming reactor comprises a fourth catalyst and being operable to receive a third part of said feed gas and to utilize at least part of said first and/or second synthesis gas streams as heating media in heat exchange within said gas heated steam methane reforming reactor, said gas heated steam methane reforming reactor being arranged for generating a third synthesis gas stream and outputting said third synthesis gas stream from said reforming section as at least part of said combined synthesis gas stream.

7. The chemical plant according to claim 1, wherein said reforming section furthermore comprises a gas heated steam methane reforming reactor upstream of said autothermal reforming reactor, wherein said gas heated steam methane reforming reactor comprises a fourth catalyst and being operable to utilize at least part of said second synthesis gas stream as heating media in heat exchange within said gas heated steam methane reforming reactor, said gas heated steam methane reforming reactor being arranged to receive said second part of said feed gas and to provide a partially reformed second feed gas, and wherein the partially reformed second feed gas is led to the autothermal reforming reactor.

8. The chemical plant of claim 7, wherein said gas heated steam methane reforming reactor is further operable to utilize at least part of said first synthesis gas stream as heating media in heat exchange within said gas heated steam methane reforming reactor.

9. The chemical plant according to claim 1, wherein said post processing unit is a post conversion unit having an inlet for allowing addition of heated CO₂ to the combined synthesis gas stream upstream the post conversion unit and housing a fifth catalyst active for catalyzing steam methane reforming, methanation and reverse water gas shift.

10. The chemical plant according to claim 1, wherein said post processing unit is a water gas shift unit arranged to carry out the water gas shift reaction.

11. The chemical plant according to claim 1, wherein said downstream section comprises gas separation unit(s) arranged to separate a stream of substantially pure CO₂, H₂, and/or CO from said intermediate synthesis gas, thereby providing a refined synthesis gas.

12. The chemical plant according to claim 1, wherein said downstream section comprises an ammonia reactor to convert said intermediate synthesis gas or said refined synthesis gas to ammonia, a methanol reactor to convert said intermediate synthesis gas or said refined synthesis gas to methanol, or a Fischer-Tropsch reactor to convert said intermediate synthesis gas or said refined synthesis gas to a mixture of higher hydrocarbons.

13. A process for producing a chemical product from a feed gas comprising hydrocarbons, in a chemical plant comprising a reforming section, said reforming section comprising an electrically heated reforming reactor housing a first catalyst, an autothermal reforming reactor in parallel with said electrically heated reforming reactor, said autothermal reforming reactor housing a second catalyst, said process comprising the steps of:
inletting a first part of said feed gas to said electrically heated reforming reactor and carrying out steam methane reforming to provide a first synthesis gas stream,
inletting a second part of said feed gas to said autothermal reforming reactor, and carrying out reforming to provide a second synthesis gas stream,
outputting a combined synthesis gas stream comprising at least part of said first and/or second synthesis gas streams from said reforming section,
optionally, in a post processing unit downstream said electrically heated reforming reactor and said autothermal reforming reactor, post processing said combined synthesis gas stream to provide a post processed synthesis gas stream,
separating said combined synthesis gas stream or said post processed synthesis gas stream into a water condensate and an intermediate synthesis gas in a water separation unit downstream said post processing unit, and
providing said intermediate synthesis gas to a downstream section arranged to receive the intermediate synthesis gas and to process the intermediate synthesis gas to a chemical product and an off-gas.

14. The process according to claim 12, wherein said electrically heated reforming reactor comprises a pressure shell housing an electrical heating unit arranged to heat said first catalyst, wherein said first catalyst comprises a catalytically active material operable to catalyze steam reforming of said first part of said feed gas, wherein said pressure shell has a design pressure of between 5 and 45 bar,
a heat insulation layer adjacent to at least part of the inside of said pressure shell, and
at least two conductors electrically connected to said electrical heating unit and to an electrical power supply placed outside said pressure shell,
wherein said process further comprises the steps of:
pressurizing said first part of said feed gas to a pressure of between 5 and 45 bar, upstream said electrically heated reforming reactor,
passing an electrical current through said electrical heating unit thereby heating at least part of said first catalyst to a temperature of at least 800° C.

15. The process according to claim 13, further comprising:
providing fuel to a fired heater unit upstream said autothermal reforming reactor, thus preheating said second part of said feed gas, and
recycling at least part of said off-gas from said downstream section as fuel to the fired heater unit.

16. The process according to claim 13, wherein said reforming section furthermore comprises a fired steam methane reforming reactor upstream said autothermal reforming reactor, wherein said steam methane reforming reactor comprises one or more tubes housing a third catalyst, wherein said fired steam methane reforming reactor comprises one or more burners for providing heat for the steam methane reforming reaction within said one or more tubes, said process furthermore comprising the steps of:
inletting said second part of the feed gas into said fired steam methane reforming reactor, and carrying out steam methane reforming within tubes of said fired reforming reactor to provide a partially reformed second feed gas,
providing said partially reformed second feed gas to said autothermal reforming reactor, and
recycling at least part of said off-gas from said downstream section as fuel to the one or more burners of the fired steam methane reforming reactor.

17. The process according to claim 13, wherein said reforming section furthermore comprises a gas heated steam methane reforming reactor in parallel to the combination of said electrically heated reforming reactor and said autothermal reforming reactor, wherein said gas heated steam methane reforming reactor comprises a fourth catalyst, said process furthermore comprising the steps of:
inletting a third part of said feed gas into said gas heated steam methane reforming reactor,
utilizing at least part of said first and/or second synthesis gas streams as heating media in heat exchange within said gas heated steam methane reforming reactor,
generating a third synthesis gas stream over the fourth catalyst within the gas heated steam methane reforming reactor, and
outputting said third synthesis gas stream from said reforming section as at least part of said combined synthesis gas.

18. The process according to claim 13, wherein said reforming section furthermore comprises a gas heated steam methane reforming reactor upstream of said autothermal reforming reactor, wherein said gas heated steam methane reforming reactor comprises a fourth catalyst, said process further comprising the steps of:
inletting said second part of the feed gas into said gas heated steam methane reforming reactor, and carrying out steam methane reforming within said fired reforming reactor to provide a partially reformed second feed gas,
providing said partially reformed second feed gas to said autothermal reforming reactor, and
utilizing at least part of said second synthesis gas streams as heating media in heat exchange within said gas heated steam methane reforming reactor.

19. The process according to claim 18 further comprising the step of:
utilizing at least part of said first synthesis gas stream as heating media in heat exchange within said gas heated steam methane reforming reactor.

20. The process according to claim 13, wherein said post processing unit is a post conversion unit housing a fifth catalyst active for catalyzing steam methane reforming, methanation and reverse water gas shift reactions, wherein said process furthermore comprises the step of inletting heated CO₂ to the combined synthesis gas stream upstream post conversion unit.

21. The process according to claim 13, wherein said post processing unit is a water gas shift unit and the step of post processing said combined synthesis gas stream comprises carrying out the water gas shift reaction.

22. The process according to claim 13, wherein said process comprises separating a stream of substantially pure CO₂, H₂, and/or CO from said intermediate synthesis gas, thereby providing a refined synthesis gas, in one or more gas separation unit(s) of said downstream section.

23. The process according to claim 13, wherein the first part of the feed gas is about 5-20 vol % of the feed gas.

24. The process according to claim 17, wherein the first part of the feed gas is about 5-10 vol % of the feed gas and the third part of the feed gas is about 5-10 vol % of the feed gas.

25. The process according to claim 13, wherein said process further comprises: converting said intermediate synthesis gas to ammonia in an ammonia reactor of said downstream section, to convert said intermediate synthesis gas to methanol in a methanol reactor of said downstream section, or to convert said intermediate synthesis gas to a mixture of higher hydrocarbons in a Fischer-Tropsch reactor.
